(19) United States
(12) Patent Application Publication
(10) Pub. No.: US 2023/0211135 A1
(43) Pub. Date: Jul. 6, 2023

(54) Method and device to improve hydrocephalus shunt systems

(71) Applicant: Victor Popa-Simil, Los Alamos, NM (US)

(72) Inventor: Victor Popa-Simil, Los Alamos, NM (US)

(21) Appl. No.: 18/092,270

(22) Filed: Dec. 31, 2022

Related U.S. Application Data
(60) Provisional application No. 63/295,941, filed on Jan. 2, 2022.

(51) Int. Cl. A61M 27/00

(52) U.S. Cl. CPC A61M 27/006; A61M 2205/0238; A61M 2205/0244; A61M 2205/3584; A61M 2205/3344; A61M 2205/3368

(57) ABSTRACT

A method and device used to improve the operation of a hydrocephalus shunt system based on the use of alpha and beta radioactive isotopes implanted in the critical zones of the shunt in order to prevent the deposition of organic matter such as blood cells, tissue, or bacteria, thereby clogging the system and causing malfunction.

### CROSS REFERENCE TO RELATED APPLICATIONS

This application claims priority of 63/295,941 from Jan. 2, 2022.

A method and device used to improve the operation of a hydrocephalus shunt system based on the use of alpha and beta radioactive isotopes implanted in the critical zones of the shunt that prevents the deposition of organic matter such as blood cells, tissue, or bacteria, thereby clogging and putting the system into malfunction.

### STATEMENT REGARDING FEDERALLY SPONSORED R&D

This invention was made with NO Government support.

### NAMES OF PARTIES TO A JOINT RESEARCH AGREEMENT

This work was part of research of the mentioned inventors.

### BACKGROUND OF THE INVENTION

### 1. Field of the Invention

The present invention relates to a method and device to prevent clogging of hydrocephalus stent's active parts and extend the good operation duration, preventing malfunctions that are corrected by invasive emergency surgery.

There are various versions of stents used to regulate the pressure inside hydrocephalus liquid in the brain; all of them rely on a pressure sensor and a release valve. Due to the presence of various organic matter in this liquid, after a short period, this matter clogs the pressure sensor or the actuating valve, triggering malfunctions.

The system consists of a radioactive alpha and beta emitter material implanted in the vicinity of sensitive parts at a distance such as to allow radiation to be present in the critical volume of CSF to soften the fluid and prevent organic matter deposition on critical surfaces such as the valve seat and pressure sensor entry. A secondary implant of a non-radioactive material is applied over the radioactive isotope implant, preventing its physical diffusion and leakage into the liquid.

Another layer is deposited having hydrophobic properties, being bio-compatible, and having a high resistance to wear.

The method consists of a set of procedures to acquire the desired implantation depth for each layer to ensure optimum functionality, such as the main radiation path takes place inside the hydrocephalus liquid. It is desired that the interaction between the radiation and liquid will drive radiolysis products in the liquid that react with dissociated water molecule free radicals, creating shorter organic compound with saturated molecular bounds that are unable to have the necessary affinity to clog mechanisms within the shunt.

### DESCRIPTION OF THE PRIOR ART

A shunt allows individuals to lead full lives, but like any other long-term medically implanted device, it can fail. A shunt is said to have failed when any complication of the treatment of hydrocephalus requires surgery.

Symptoms of a shunt malfunction may be obvious, redness over the shunt, headache, sleepiness, vomiting, or visual changes. Symptoms may also be subtle, change in behavior, change in school performance. Typically, shunt malfunction is suspected when one or more of the symptoms of hydrocephalus observed prior to shunting return. When complications do occur, further testing is required and patient may need to undergo a shunt revision, which is an operation to replace the section of the shunt that is no longer working. When shunt malfunction is suspected, it is critical for the user to seek medical assistance immediately.

The most common shunt complications are malfunction and infection are:

a) Shunt Malfunction or Shunt Failure, is a partial or complete blockage (obstruction) of the shunt that causes it to function intermittently or not at all. When a blockage occurs, cerebrospinal fluid (CSF) accumulates and can result in symptoms of untreated hydrocephalus. A shunt blockage from blood cells, tissue, or bacteria can occur in any part of the shunt. Both the ventricular catheter (the portion of the tubing placed in the brain) and the distal part of the catheter (the tubing that drains fluid to another part of the body) can become blocked by tissue.

Shunts are very durable, but their components can become disconnected or fractured as a result of wear or as a child grows. Occasionally they dislodge from where they were originally placed. Breakage causes a total or partial interruption in the shunt pathway, which may obstruct fluid flow and add resistance to the system. A disconnection may occur, but the formation of scar tissue around the subcutaneous catheter may still allow fluid to flow. Migration may also alter shunt function, causing catheters to move to locations that may restrict flow. Rarely, a valve will fail because of a mechanical malfunction. With a programmable valve and experiencing symptoms of a shunt malfunction, if it can be determined that the shunt is still capable of flow, your doctor may adjust your setting to avoid an operation. This failure is intended to be addressed by the present invention.

b) Shunt Infection is usually caused by a person's own bacterial organisms and is not acquired from other children or adults who are ill. The most common infection is Staphylococcus Epidermidis, which is normally found on the surface of a person's skin and in the sweat glands and hair follicles deep within the skin. This type of infection is most likely seen one to three months after surgery but can occur up to six months or more after the placement of a shunt. Patients treated with ventriculo-atrial (VA) shunts may develop a more serious infection, which may enter the bloodstream. When a shunt infection occurs, the standard treatment is the surgical removal of all of the shunt hardware. An External Ventricular Drain (EVD) is surgically placed to manage the hydrocephalus while the shunt is removed and the infection is being treated. The patient remains in the hospital while the infection is being treated with antibiotics, approximately 10-14 days. When the infection has cleared, the new shunt is implanted surgically.

### c) Other Shunt Complications

C1. Over drainage causes the ventricles to decrease in size and may create slit-like ventricles as a result of excessive drainage of CSF. Slit ventricles are most commonly found in young adults who have been shunted since childhood. Some people have slit ventricles but do not experience any symptoms. Symptoms include typical signs of shunt malfunction and often are provoked by standing and relieved by lying horizontal, although if they persist for a long time, they may lose this distinctive characteristic.
C2. Slit-ventricle syndrome (SVS) may be diagnosed when people have slit ventricles and experience specific symptoms. A particular symptom of SVS is severe intermittent headaches that are often relieved when lying down. Imaging studies are required to determine SVS, which is typically indicated by smaller than normal ventricles. Most shunt manufacturers have shunt hardware designed to help decrease slit-ventricle syndromes.
C3. Under drainage causes the ventricles to increase in size and can fail to relieve the symptoms of hydrocephalus. To restore a balanced flow of CSF it may be necessary to place a new shunt with a more appropriate pressure setting. For those who have externally adjustable or programmable valves, the balance of flow may be restored by re-setting the opening pressure. Symptoms of under drainage include headaches with increasing frequency and severity, which are often worse on waking in the morning.
Also, vomiting and dizziness may be signs of under drainage. In older children, symptoms may include increased irritability, ‘laziness’, poor or disruptive school performance, or even more antisocial activity.
C4. Adjustment to accommodate patient growth. In children, it may be necessary to modify or revise a shunt in order to adjust for patient growth. For infants implanted at birth, the ventricular catheter may have to be changed around two years to accommodate brain growth. For children, the shunt may need to be revised as the child grows. For instance, as the child gets taller, the tubing from the head to the peritoneal cavity (VP shunt) may need to be replaced with a longer tube. However, neurosurgeons try to minimize the need for revisions. For instance, excess length of VP shunt tubing is placed in the peritoneal cavity with the distal catheter. The catheter slowly pulls out of the peritoneal cavity as the child gets taller. For this reason, regular physician follow-up for shunt assessment and maintenance is crucial, particularly in growing children.
C5. Subdural hematoma occurs if blood from broken vessels becomes trapped between the brain and skull. This is most common in older adults with normal pressure hydrocephalus (NPH) and requires surgery to correct.
C6, Multiloculated hydrocephalus is a loculated (isolated) CSF compartment in the brain that is enlarged but not connected to the ventricular system. It may be caused by birth trauma, neonatal intraventricular hemorrhage, ventriculitis (infection of the ventricle), shunt related infection, over drainage or other conditions. This complication may be difficult to identify because it is typically seen in infants and children who may be neurologically compromised. Surgical treatments include placement of multiple shunts, ventricular catheters with multiple perforations or openings, endoscopic or craniotomy to fenestrate (open) the intraventricular loculations.
C7. Material degradation. Originally, barium sulfate was mixed with silicone to allow shunt catheters to be visible on x-ray. These barium sulfate crystals eventually dissolved, making the tubing surface rough. Tissue in-growth to the roughened surface caused binding of the tubing at specific locations which promoted breakage or deterioration of the tubing. Shunt tubing design has been changed and clear silicone elastomer now covers the surface greatly reducing the possibility of this to occur.
C8. Seizures sometimes occur in people with hydrocephalus. There is no correlation between the number of shunt revisions or the site of shunt placement and an increased risk of developing seizures. Past studies have shown that children with hydrocephalus who have been treated with a shunt and who also have a significant cognitive delay or motor disability are more likely to experience seizures than those without cognitive or motor delays. Studies have also indicated that seizures are not likely to occur at the time of shunt malfunction and that the most likely explanation of seizure disorder is the presence of associated malformations of the cerebral cortex.
C9. Abdominal complications in the abdomen can occur in people with hydrocephalus treated with a shunt. Shunt complications that develop in the peritoneum or abdominal area include peritoneal pseudocysts, lost distal catheters, bowel perforations, and hernias. The peritoneum or abdominal area is the most popular site for distal catheter implantation. Although ventriculoperitoneal (VP) shunts do not have fewer complications than ventriculoatrial (VA) shunts, the complications are less severe.
C10. Infection of a ventriculoatrial (VA) shunt leads to a bloodstream infection and is more concerning than an infection of a ventriculoperitoneal (VP) shunt. Rarely, chronic infection can cause kidney damage or life-threatening damage to the lungs and heart. VA shunts do not fail any more often than VP shunts, but because their complications may be more serious, they are reserved for special circumstances. Complications for Ventriculoatrial (VA) shunts have been associated with pulmonary hypertension, pulmonary tree embolization, and shunt nephritis (an inflammation within the kidney).
C11. Rare Complications include intestinal volvulus (twisting) around the shunt catheter, formation of encapsulated intra-peritoneal CSF compartments, or development of reactions to the implanted materials.
   D. There are some diagnostic and treatment as follows:
D1. Shunt flow studies, which also may be referred to as a shunt patency study or shunt gram, is a study to determine in real-time if CSF is flowing through the shunt system. By injecting a small volume of contrast dye or a radiotracer into the shunt reservoir, the flow of CSF through the catheters and valve can be measured.
D2. Shunt Tap is a diagnostic test to screen for infection and confirm that the shunt is still functioning. The area of skin overlying the shunt reservoir is cleansed with a sterile antibacterial solution. For a shunt tap, a small needle is used to pierce the skin and access the shunt reservoir/antechamber. The doctor often collects and sends a CSF sample for investigation to rule out any source of shunt-related infection.
D2. External Ventricular Drain (EVD) is a treatment that allows the temporary drainage of CSF from the lateral ventricles of the brain, or lumbar space of the spine, into an external collection bag. An EVD drains the CSF by using a combination of gravity and intercerbral pressure. The drainage rate depends on the height at which the EVD system is placed relative to the patient's anatomy relieving raised intracranial pressure (ICP).
EVDs are often used to relieve elevated ICP, drain infected CSF, drain bloody CSF or blood after surgery or hemorrhage, and monitor the flow rate of CSF.
D3. Intracranial Pressure Monitoring (ICP) is a diagnostic test that helps your doctors determine if high or low CSF pressure is causing your symptoms. When the cause of a headache has resisted every other diagnostic measure, the surgeon may recommend admission to the hospital for ICP monitoring. ICP monitoring requires a surgical procedure. Your surgeon will make a small hole, called a burr hole, in the skull and a small pressure monitor is inserted through the brain and into a lateral ventricle to measure the ICP. Your pressures are recorded continuously and provide critical guidance for therapy.

There are known for long time that using radioactive stents may result in an extension of period between malfunctions. There is increasing interest in the use of vascular irradiation, from an internally introduced radioactive source to control restenosis after balloon angioplasty.

Developing models both experimental and theoretical, of the kinetics of radiation-induced smooth muscle cell (SMC) inactivation and regrowth, as a first step toward optimizing the design of clinical vascular irradiation. Both animal experiments and early clinical studies appear to show promising results in this regard. We consider various mechanistic interpretations of the experimental and clinical observations that doses of 12-20 Gy appear to be efficacious in preventing restenosis. We develop and investigate simple models, both experimental and theoretical, of the kinetics of radiation-induced smooth muscle cell (SMC) inactivation and regrowth, as a first step toward optimizing the design of clinical vascular irradiation, as dr. D J Brenner, R C Miller, E J Hall shown in their paper: “The radiobiology of intravascular irradiation” published in Int J Radiat. Oncol. Biol. Phys. 1996 Nov. 1; 36(4):805-10, who was using in vitro models of human SMCs, to investigate the relative radio sensitivity of SMCs compared with endothelial cells and measure the dose-dependent ability of SMCs to repopulate a denuded region in a confluent layer of cells. They found that doses>20 Gy, which would be required to completely eliminate the SMC population which has the potential to cause restenosis, are too large to be practical because of the unacceptable risk of late complications. However, doses that can be practically given in vascular irradiation (<20 Gy) will certainly delay restenosis by 1-3 years, with larger doses producing longer delays. Whether such doses can avert restenosis permanently is unclear, as permanent prevention at realistic doses depends critically on the assumption that those SMCs which survive irradiation have a significantly limited capacity for proliferation.

In the paper “Low-dose radioactive endovascular stents prevent smooth muscle cell proliferation and neointimal hyperplasia in rabbits”, published in: Circulation, 1995 Sep. 15; 92(6):1570-5, authors C Hehrlein 1, C Gollan, K Dönges, J Metz, R Riessen, P Fehsenfeld, E von Hodenberg, W Kübler teaches Restenosis induced by smooth muscle cell (SMC) migration and proliferation and neointimal thickening limits the clinical success of balloon angioplasty and stent implantation. In this study, the long-term effect of endovascular irradiation via low-dose radioactive stents on neointima formation was compared with conventional stent implantation in a rabbit model. Methods and results: Palmaz-Schatz stents were made radioactive in a cyclotron. The stents had a very low activity (maximum, 35 micro-Ci), and thus, manipulation did not require extensive radiation protection. One, 4, 12, and 52 weeks after the implantation of nonradioactive stents and radioactive stents in rabbit iliac arteries, neointimal thickening was analyzed by quantitative histomorphometry. Immunostaining for endothelial cell von Willebrand factor, macrophages, SMC alpha-actin, collagen type I, and proliferating cell nuclear antigen (PCNA) was performed to determine radiation-induced changes in the arterial wall. SMC proliferation was quantified by computer-assisted cell counting of PCNA-immunoreactive cells. Neointima formation was markedly suppressed by the implantation of radioactive stents in a dose-dependent fashion at all observed time points. At peak proliferative activity of SMCs 1 week after nonradioactive stent implantation, 30+/−2% of SMCs in the neointima were proliferating, compared with 0.5+/−0.1% of SMCs after implantation of stents with an initial activity of 35 microCi (P<0.001). The neointima covering radioactive stents was characterized by decreased smooth muscle cellularity and increased extracellular matrix formation. Further, we observed a delayed endothelialization depending on the radiation dose. No difference in vascular thrombosis was found after nonradioactive and radioactive stent implantation. The results of this study clearly indicate that low-dose radioactive endovascular stents potently inhibit SMC proliferation and neointimal hyperplasia in rabbits.

In a paper in Circulation, by 1996 Feb. 15; 93(4):641-5. entitled “Pure beta-particle-emitting stents inhibit neointima formation in rabbits” written by C Hehrlein 1, M Stintz, R Kinscherf, K Schlösser, E Huttel, L Friedrich, P Fehsenfeld, W Kübler they teach about experimental evidence exists that neointimal hyperplasia after angioplasty is inhibited by gamma-irradiation of the treated arteries. A beta-particle radiation is absorbed in tissue within a shorter distance away from the source than gamma-radiation and may be more suitable for localized vessel irradiation. This study outlines a method to implant a beta-particle-emitting radioisotope (₃₂P; half-life, 14.3 days) into metallic stents. The effects of these stents on the inhibition of neointimal hyperplasia was compared with conventional stents in a rabbit model. The isotope ₃₂P was produced by irradiation of red amorphous phophorus (₃₁P) with neutrons and was implanted into Palmaz-Schatz stents (7.5 mm in length) after being kept apart from ₃₁P in a mass separator. The radioisotope was tightly fixed to the stents, and the ion implantation process did not alter the surface texture. Stent activity levels of 4 and 13 microCi were chosen for the study. Four and 12 weeks after placement of conventional stents and ₃₂P-implanted stents in rabbit iliac arteries, vascular injury and neointima formation were studied by histomorphometry. Immunostaining for smooth muscle cell (SMC) alpha-actin was performed to determine SMC cellularity in the neointima. SMCs were quantified by computer-assisted counting of alpha-actin immunoreactive cells. Endothelialization of the stents was evaluated by immunostaining for endothelial cell von Willebrand factor. No difference in vessel wall injury was found after placement of conventional and 32P-implanted stents. Neointima formation was potently inhibited by 32P-implanted stents only at an activity level of 13 microCi after 4 and 12 weeks. Neointimal SMC cellularity was reduced in ₃₂P-implanted stents compared with conventional stents. Radioactive stents were endothelialized after 4 weeks, but endothelialization was less dense than in conventional stents. They concluded that neointima formation in rabbits is markedly suppressed by a beta-particle-emitting stent incorporating the radioisotope ₃₂P. In this model, a dose-response relation with this type of radioactive stent was observed, indicating that a threshold radiation dose must be delivered to inhibit neointima formation after stent placement over the long term. The problem with ₃₂P isotope is its 2 weeks halving time being possible of being effectively used for about 3 months, then its radioactivity becomes smaller than 1%, fact that makes clear for us that longer lives radioisotopes have to be used.

In the publication Semin Interv Cardiol from 1997 June; 2(2):109-13 a sintesys entitled “Advantages and limitations of radioactive stents”, was written by C Hehrlein 1, W Kübler from Department of Cardiology, University of Heidelberg, Germany, where they state that the concept of radioactive stents was initiated to prevent restenosis after angioplasty in patients with coronary artery disease. We review the modes of fabrication, dosimetry and the biological effects of radioactive stents. Radioactive stents deliver ionizing radiation continuously at very low-dose rates according to the half-life of the incorporated radioisotopes. The activity levels of radioactive stents are up to 10,000 times lower than activity levels of sources used for catheter-based vascular brachytherapy. Radioactive stents allow uniform dose distribution and precise dosimetry because of the direct source contact with the circumference of the vessel. Animal studies show that these stents can potently inhibit smooth muscle cell proliferation and neointimal hyperplasia. A persistent inhibition of neointimal hyperplasia appears to be dose dependent. Local or systemic side effects related to the irradiation were not observed. A limitation of radioactive stents could be the dose-dependent delay in stent endothelialization which, however, did not cause thrombotic vessel occlusion in animal experiments. Whether a delay in stent endothelialization is associated with an increased rate of occlusive stent thrombosis in humans requires further studies. From here we understand that there is possible to apply this principle at various other devices exposed to body fluids. In the publication J Invasive Cardiol, by 2000 March; 12(3):162-7. Published a paper entitled “The impact of stent design and delivery upon the long-term efficacy of radioisotope stents” where T A Fischell, Director, Heart Institute at Borgess Medical Center, 1521 Gull Road, Kalamazoo, Mich. 49001, USA, and his collaborators, C Hehrlein, R E Fischell, D R Fischell teach about both gamma and beta irradiation delivered via a radioactive catheter-based line source have been shown to have efficacy in reducing restenosis. However, these catheter-based treatments have some limitations, including the safety of handling sources ranging from 30 mCi to 500 mCi.

Alternatively, one could use a stent as the platform for local radiation delivery as a means to prevent restenosis. Experimental studies have demonstrated that stents ion implanted with the b-particle emitter ₃₂P can reduce neointima formation. Clinical evaluation of the radioisotope stent began in the fall of 1996. Dose escalation studies have now been completed in approximately 250 patients with ₃₂P, b-particle emitting stents ranging from 0.5 microCi to 24 microCi. Overall, these feasibility trials have demonstrated a clear, dose-dependent reduction of neointimal hyperplasia within the stent structure, but with an unanticipated finding of a relatively high incidence of restenosis at the stent margins. The purpose of this paper is to review the current status of radioactive stents, with an emphasis on the key elements of stent design and stent delivery that could impact the long-term efficacy of this device. W A Tan 1, C R Jarmolowski, L R Wechsler, M H Wholey from Pittsburgh Vascular Institute, University of Pittsburgh Medical Center, Pennsylvania 15232, USA discussed about “New developments in endovascular interventions for extracranial carotid stenosis”, published by Tex Heart Inst J. 2000; 27(3):273-80 where they provide an overview of recent developments in carotid interventional technique and equipment, including new stents and emboli-protection devices. The newer self-expanding stents lessen the problem of external stent compression

associated with balloon expandable stents, but precise deployment and the matching (by length) of stents to lesions remain problematic. Discuss emerging pharmacologic strategies for cerebral protection in stroke. Multiple randomized clinical trials and multicenter registries are under way to compare percutaneous with surgical strategies for the treatment of carotid stenosis. These include the evaluation of emboli protection devices, and, to a lesser degree, intravenous glycoprotein IIb/IIIa antagonists. Other clinical trials are aimed towards refining the ability to stratify patients by risk, in order to identify the subsets that would benefit most from these complex and expensive procedures.

### SUMMARY OF THE INVENTION

The present invention is a method to produce improved shunts for Shunt Malfunction or Shunt Failure and Shunt Infection presented above at item a) and b) and only indirectly affecting items c) and d). The device is a shunt where the technologic important surfaces have been treated and improved, making them immune to clogging and sterilizing the fluid down flow from the shunt.

Adding a WiFi communication inside the shunt to monitor the internal pressure and shunt operation will be another improvement of the device, making it predictable and more reliable. There are more shunt manufacturers, using about same assemblies. Before installing, after passing the quality control the parts are sent to a specialized unit that has an implanter that applies multiple isotopes implantation as an embodiment of a present invention on the specified surfaces of interest. As methodology describes first is implanted an in-depth isotope that prevents the diffusion inside material of alpha emitter isotopes. For beta emitters this is not applied because electrons range is by a factor of 10 larger and micron range diffusion may not affect too much the general electron density in the fluid volume.

After this treatment, the radioactive isotope is implanted at a controlled depth of few microns under the surface. The conditions such isotope have to meet are that the live time or halving-time to be in the year long range, but not longer than 100 years, and to have minimal spontaneous gamma or n emission, emitting a clean radiation, as another embodiment of the present invention.

The following alpha emitter isotopes have to be used:

| Izotope | Z | N | <A> | T½ | Decay | Isotope | Spin |
|---|---|---|---|---|---|---|---|
| 208Po | 84 | 124 | 207.9812457 | 2.898 y | α (99.99%) | 204Pb | 0+ |
| | | | | | β+ (.00277%) | 208Bi | |
| 209Po | 84 | 125 | 208.9824304 | 125.2 y | α (99.52%) | 205Pb | ½− |
| | | | | | β+ (.48%) | 209Bi | |
| 210Po | 84 | 126 | 209.9828737 | 138.376 d | α (99.99%) | 206Pb | 0+ |
| 210mBi | 86 | 134 | 271.31 | 3.04 × 106 y | α | 206Tl | 9− |
| 228Ra | 88 | 140 | 228.0310703 | 5.75 y | β− | 228Ac | 0+ |
| 227Ac | 89 | 138 | 227.0277521 | 21.772 y | β− (98.61%) | 227Th | 3/2− |
| 228Th | 90 | 138 | 228 | 1.9116 y | α | | |
| 232U | 92 | 140 | 232 | 68.9 y | α | 228Th | |
| 238Pu | 94 | 144 | 238 | 87.74 y | α | 234U | |
| 241Pu | 94 | 145 | 241 | 14 y | α/SF | 237U | |
| 241Am | 95 | 146 | 241 | 432.2 y | α | 237Np | |
| 242m1Am | 95 | 147 | 242 | 141 y | α | 238Np | |
| 243Cm | 96 | 147 | 243 | | α | 242Am | 5/2+ |
| 244Cm | 96 | 148 | 244.0627526 | 18.10 y | α | 240Pu | 0+ |
| 248Cf | 98 | 150 | 248.072185 | 333.5 d | α | 244Cm | |
| 249Cf | 98 | 151 | 249.0748535 | 351 y | α | 245Cm | 9/2− |
| 250Cf | 98 | 152 | 250.0764061 | 13.08 y | α | 246Cm | 0+ |
| 251Cf | 98 | 153 | 251.079587 | 900 y | α | 247Cm | ½+ |

Beta emitters that may be considered:

| Radionuclide | Half-life | | Maximum β energy [keV] |
|---|---|---|---|
| 3H | 12.3 | y | 18.6 (Tritium mobility high—not |
| | | | recommended) |
| 14C | 5730 | y | 156 (too low radioactivity—not |
| | | | recommended) |
| 85Kr | 10.8 | y | 670 (noble gas—hard to |
| | | | stabilize—but optimal if implanted) |
| 90Sr | 28 | y | 546 (has 90Y as precursor) |
| 90Y | 64 | h | 2270 (too high energy = long range) |

In spite Tritium (T) has a reasonable halving time but electron energy is low and it may be used only if substitutes the H atoms in the polymer composition as to be bound and diffusion free.

### BRIEF DESCRIPTION OF THE DRAWINGS

FIG. 1A Hydrocephalus shunt implanted in a Human body;

FIG. 1B Shunt details;

FIG. 2 Schematic diagram of a shunt valve;

FIG. 3 Main corpuscular radiation mechanisms;

FIG. 4 Chart showing beta particles LET (Linear Energy Transfer) with distance;

FIG. 5 Details on electrons stopping in matter;

FIG. 6 Details of alpha particles kinematics in air;

FIG. 7 Alpha particles kinematics details;

FIG. 8 Valve shutter mechanism details;

FIG. 9 Radioactive layer structure;

FIG. 10 Shunt with WiFi communication;

### FIGURES DETAILS

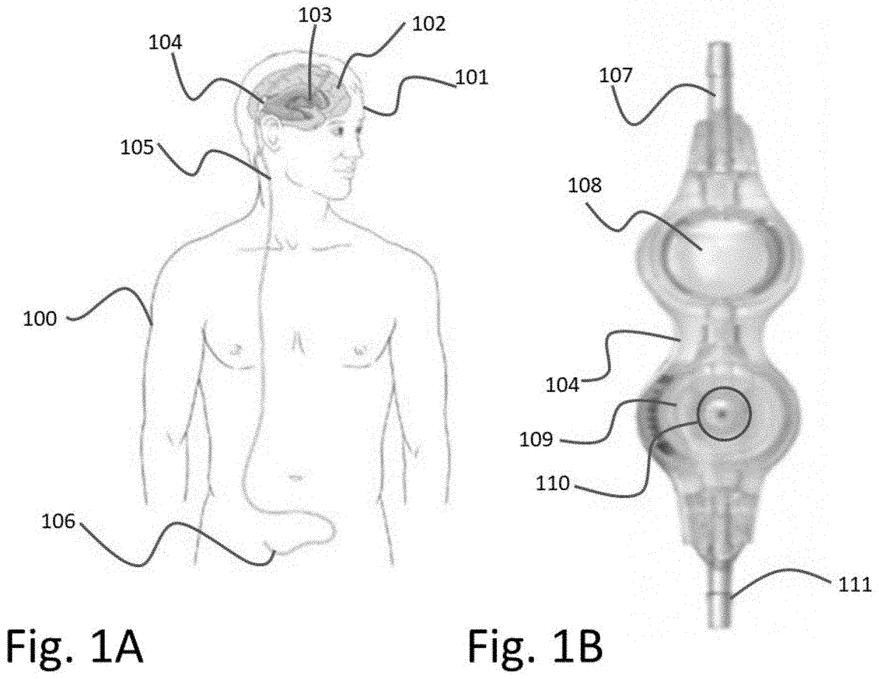

FIG. 1A Hydrocephalus stent implanted in a Human body:
100—Body;
101—Head;
102—Brain;
103—Tube inserted in hydrocephalic cavity;
104—Pressure regulator shunt;
105—plastic tube;
106—Tube discharge end in stomach.

FIG. 1B—Shunt details:
104—Pressure regulator shunt;
107—Liquid input;
108—First cavity;
109—Valve cavity;
110—Valve seat;
111—Shunt exit.

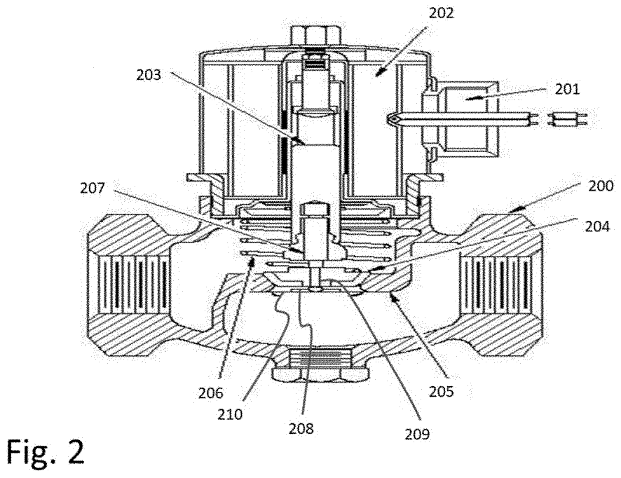

FIG. 2 Schematic diagram of a valve:
200—Valve's body;
201—Conduit opening;
202—Coil;
203—Plunger;
204—Valve stem;
205—Valve seat;
206—Plunger's spring;
207—Valve flow space;
208—Valve central lid;
209—Valve's lid rod;
210—Valve sit.

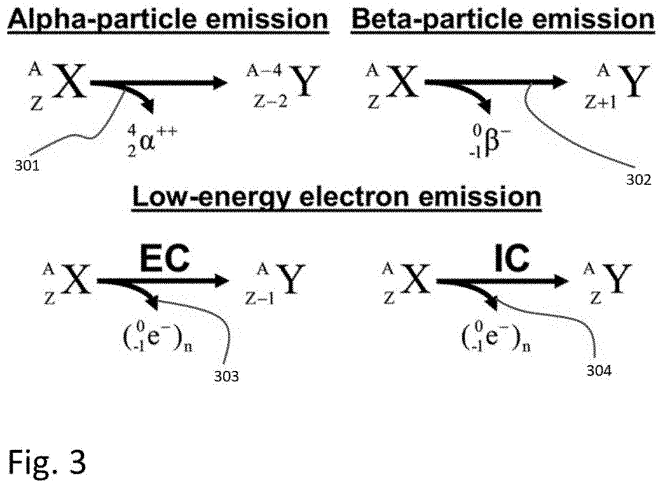

FIG. 3 Main corpuscular radiation mechanisms:
301—Alpha decay;
302—Beta decay;
303—Electron capture;
304—Ionization collision.

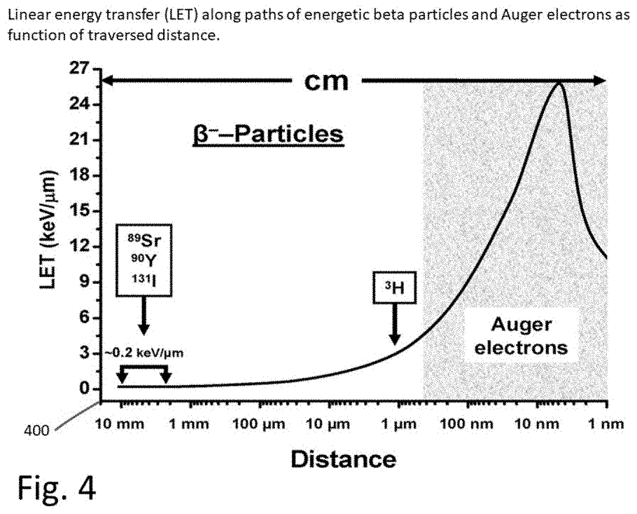

FIG. 4—Chart showing beta particles LET (Linear Energy Transfer) with distance:
400—Chart Linear Energy Transfer as function of Distance in lg. scale.

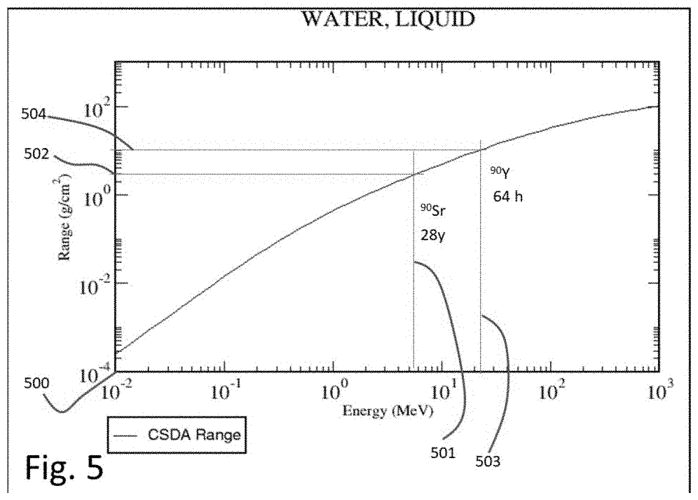

FIG. 5—Details on electrons stopping in matter:
500—Chart showing Range in g/cm2 versus particle energy in MeV 1 MeV=1.6 10\hat{ }−13 J;
   CSDA=The continuous slowing down approximation (CSDA) range represents the path length that an electron would traverse when slowing down from its original energy E. to a stop, if its rate of energy loss along the track were equal to the mean rate of energy loss. For water density=1 g/cm3 therefore may interpret ranges in cm.
501—90Sr energy of 464 keV;
502—Range in water for this energy;
506—90 Y energy of 2.3 MeV;
507—Range for 90Y is 10 cm in water.

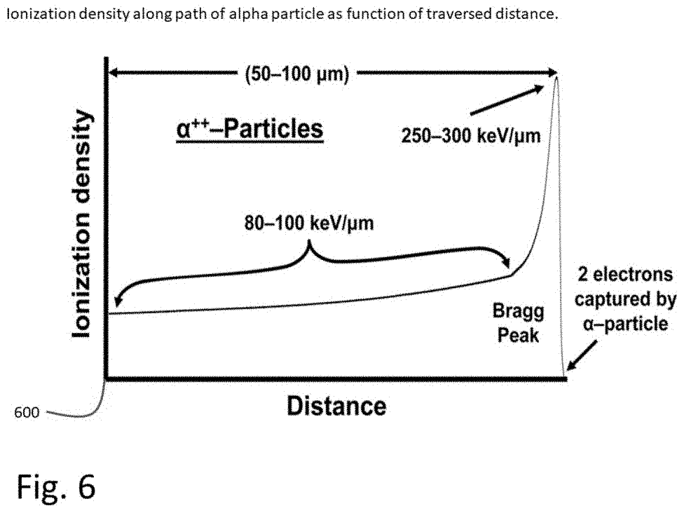

FIG. 6 Details of alpha particles kinematics in air:
600—Chart showing ionization density versus distance.

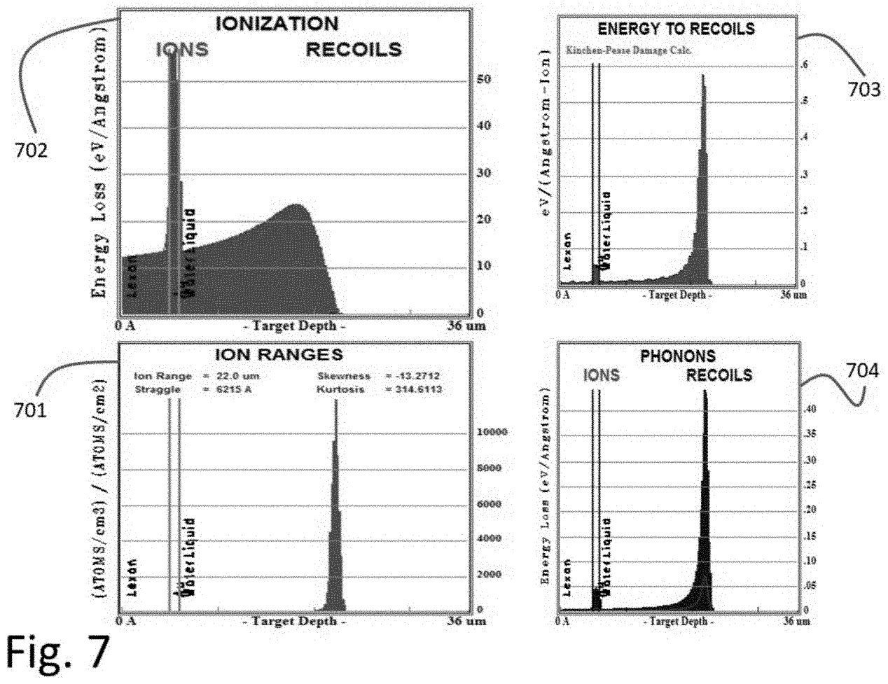

FIG. 7 Alpha particles kinematics details acting in Lexan, 5 micron coated by 2 micron of Au holding watery liquid:
701—Ion Ranges in the Lexan, Au, Water structure;
702—Alpha particle energy loss by ionization in the Lexan, Au, Water structure;
703—Alpha particles energy deposition into recoils in the Lexan, Au, Water structure;
704—Alpha particle energy deposition into phonons in the Lexan, Au, Water structure;

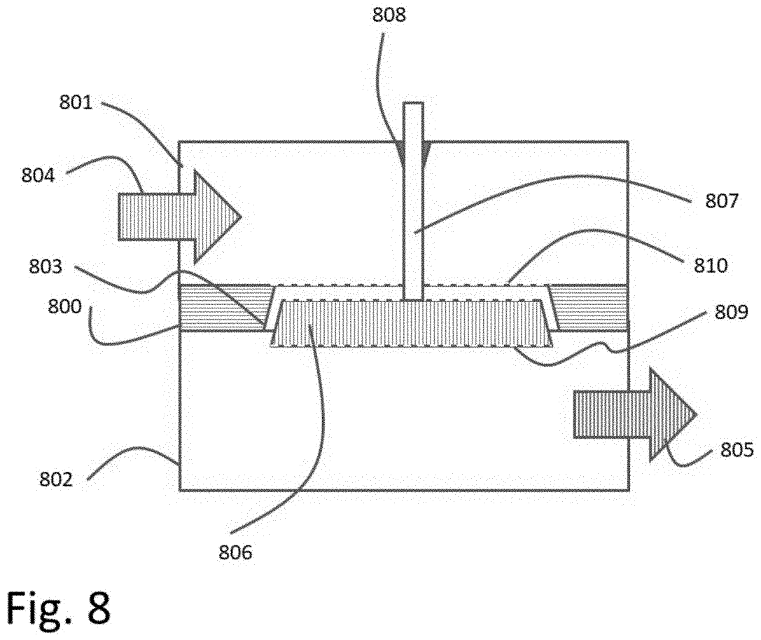

FIG. 8 Valve shutter mechanism details:
800—Valve separation structure;
801—Liquid intake cavity;
802—Liquid exhaust cavity;
803—Valve seat;
804—Liquid intake flow;
805—Liquid output flow;
806—Valve lid;
807—Valve lid's rod;
808—Protective bellow cover;
809—Radioactive surface;
810—radioactive sit surface.

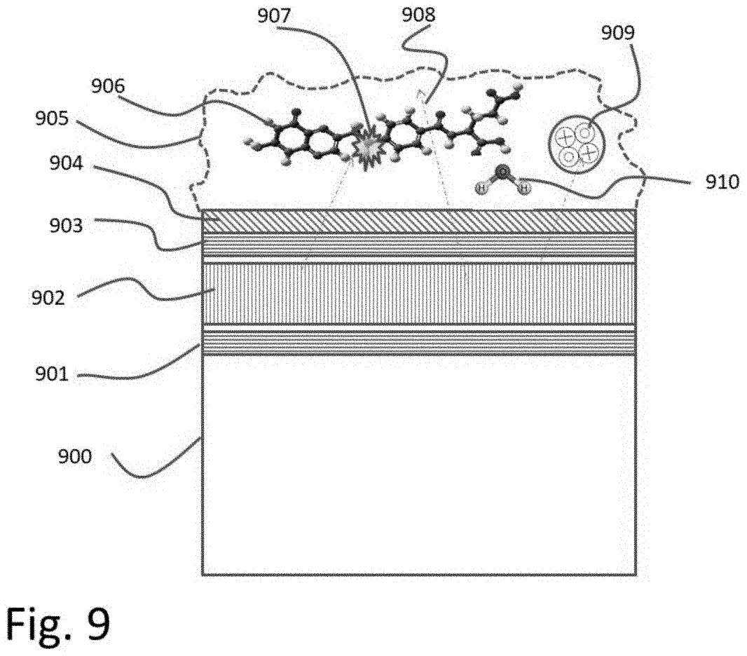

FIG. 9 radioactive layer structure:
900—Material bulk;
901—Layer to stop diffusion in bulk;
902—Radioactive isotope;
903—Layer to stop diffusion outside the bulk;
904—Chemical inert layer;
905—Hydrocephalic liquid;
906—Organic molecule;
907—Radiation split molecular bound;
908—Beta Radiation passing without interaction;
909—Alpha particles;
910—Water molecule.

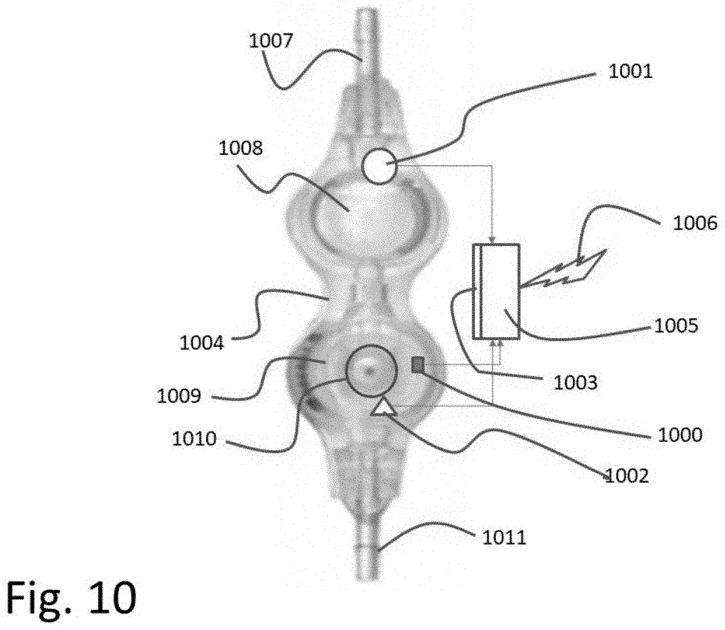

FIG. 10 Shunt with WiFi communication:
1000—Shunt's programing and control unit;
1001—Pressure measuring transducer;
1002—Valve position monitoring;
1003—Battery;
1004—Pressure regulator shunt;
1005—Communication and control micro-processor;
1006—RF signal;
1007—Liquid input;
1008—First cavity;
1009—Valve cavity;
1010—Valve seat;
1011—Shunt exit.

### DETAILED DESCRIPTION OF THE INVENTION

The inventors consider the developments in shunt radioactive protection and Wi-fi monitoring technology may be successfully used to improve the quality and duration of operation, as well to provide a reliable and safe source of information on brain pressure in various work conditions and environments, and a tool for medical researchers and professionals involved in brain functionality, physiology and pathology.

### 2. Best Mode of the Invention

FIG. 10 shows the devices in the best mode contemplated by the inventors of the use of radioactive ions implantation with biometric data acquisition and processing system which solutions and developments are embedded in the present invention.

The invention corrects the following previous deficiencies of the previous devices, as follows:
a)—Prevents parts of cerebrospinal fluid (CSF) to clog sensitive parts of the shunt, triggering its mal-operation as shunt failure or infection;
b)—Makes a system that gives a quasi-real time data on intracranial pressure (ICP) that may be correlated with other internal and external parameters to better understand the mechanisms that are involved;
c)—Is easy, adaptable to new types of shunts, and appears for shunt manufacturers as another stage in shunt production;
d)—It is versatile, allowing various types of radioisotopes to be used, inside the limits of safety for the patient and physicians;
e) Improves the warning and alert to the health provider, by detecting any anomalous evolution of the patient, based on customized data sets.

The best application of the invention is explained in FIGS. 8-10 and done by the system presented in FIG. 10. The system allows multiple active modes, with various technical combinations of ICP, as function of customized patient conditions and presentation of the integrated data, in such manner that each participant evolution to be possible to be analyzed in the smallest detail.

### 3. How to Make the Invention

As can be amply seen from the drawings the procedure includes a device that is made of a micro-processor board for data acquisition, mainly absolute pressure, and differential pressure on the shunt valve, temperature, liquid's electric conductivity, and shunt valve control signal and position, and possible other electric or magnetic signal additionally acquired from the brain, from which based on calibration the liquid flow and brain activity may be calculated. The wi-fi communication system assures data monitoring and in some cases shunt operation reprograming and optimization.

The method to produce a more robust shunt using radioactive isotopes relies on the fact that at interaction between radiation and matter radiolysis process occurs that breaks molecular bonds producing free radicals, that further may recombine producing shorter molecules and recombine, fulfilling open bounds/valences modifying the surface tension and adhesion forces and making cerebrospinal fluid (CSF) more fluid and less likely to deposit and clog the shunt's technological surfaces.

The method has the following steps:
a)—Determine in cooperation with shunt manufacturer the surfaces of interest to be implanted;
b)—The manufacturer delivers the parts of interest specifying the materials used;
c)—The isotope of interest and type of charged particle is established;
d)—Radiation transport code is applied to calculate radiation dose distribution inside the volume and outside the stent in the head area and around, confirming that all safety measures have been considered and applied;
e) If the selected charged particle is:
   a. An alpha particle the procedure of implantation has the following steps:
      i. Implant first a diffusion stopper material at a depth of several microns (that can be Au, Ag, W, etc.);
      ii. Implant the radioactive isotope with a range with 1-2 microns smaller than the previous implantation;
      iii. Implant another diffusion stopper layer at a range from surface down to the upper edge of the radioactive isotope layer;
      iv. Make a coating of few microns of a material chemically inert and with good properties for wear, corrosion, abrasion and affinity inside cerebrospinal fluid (CSF);
   b. A beta (electron) particle:
      i. Due to larger range of electrons the inside diffusion blocker layer is not needed except special circumstances when other considerations recommends it;
      ii. All the rest of steps remain the same;
f) Quality assurance (QA) methods are applied to certify the conformity of the work with the previous planning and computer simulations that may use, but not limited to the following techniques:
   a. Charged particle and associated X, gamma spectrometry;
   b. Rutherford Back-Scattering (RBS);
   c. Micro-profile measurements;
   d. Autoradiography, where is possible;
g) With the QA certificate the parts are delivered to manufacturer to assembly them and perform the final QA measurements and calibration of the whole system before being implanted inside patient's skull;
h) Before ending the surgery the final tests of the system are made, certifying that the entire parameter set is according with the plan and overall system QA certificate may be released;
i) Based on calibrations made by manufacturer the operation data acquisition and processing starts and the normal usage starts.

Together the method and device is aimed to assure a long period of good operation of the stent, while each patient monitoring will add to database helping the R&D effort in the brain and neuro-science, s identifying best operation pressures for various brain regimes.

### DETAILED DESCRIPTION OF THE FIGURES

FIG. 1A—Shows a hydrocephalus shunt implanted in a Human body, 100, where the main part is implanted inside Head, 101, near the Brain, 102, having a tube, 103, inserted in hydrocephalic cavity. The pressure regulator shunt, 104, discharges the excess fluid using a bio-compatible plastic tube, 105, with the tube discharge end, 106, in stomach from where the liquid is further eliminated.

FIG. 1B shows a detail on shunt, 104, that acts as a intracranial pressure (ICP) regulator by taking in its liquid input, 107, cerebrospinal fluid (CSF) in its first cavity, 108, measuring its pressure and passing it in valve cavity, 109, placed on a valve seat, 110, that when ICP is higher than normal valve opens, releasing ICF into shunt exit, 111, tube and from there in the stomach.

FIG. 2 shows a Schematic diagram of a valve where valve's body, 200, is made of bio-compatible plastics, with null-buoyancy inside the head, being actuated by an electromagnetic coil, 202, powered by electric cables via a conduit opening, 201, bringing power from a battery and control device. The coil actuates a plunger, 203, that pushes valve's stem, 204, moving the valve's central lid, 208, up and down from the valve's seat, 205, opening and closing valve flow space, 210. The plunger, 203, is connected via an extension rod, 207, and a smaller in diameter valve's lid rod, 209, to the valve lid, 208, compressing between a plunger's spring, 206.

FIG. 3 shows briefly main corpuscular radiation mechanisms, where alpha particles which are He nuclei are the result of Alpha decay, 301, the electrons result from beta decay, 302, or from electron capture, 303, or ionization collision, 304, and any of these reactions are used in our application.

FIG. 4 presents a chart showing beta particles LET (Linear Energy Transfer) with distance where in chart, 400, LET is given as function of distance in lg. scale for the radiation energy specific to various isotopes.

FIG. 5 gives more details on electrons stopping in matter, with respect to CSDA (The continuous slowing down approximation (CSDA)) range represents the path length that an electron would traverse when slowing down from its original energy E to a stop, if its rate of energy loss along the track were equal to the mean rate of energy loss. For water density=1 g/cm³ therefore may interpret ranges in cm, in chart, 500, showing Range in g/cm² versus particle energy in MeV (1 MeV=1.6 10⁻¹³ J). The stopping range for ⁹⁰Sr, 501, which has a maximum energy of 464 keV, showing range in water, 502, for this energy, and for ⁹⁰Y, 506, with maximum energy of 2.3 MeV, showing range in water, 507, for ⁹⁰Y.

FIG. 6 shows some details of alpha particles kinematics in air, where is given a chart showing ionization density versus distance, 600.

FIG. 7 presents more alpha particles kinematics details as it seems that these particles having a shorter range in water may be more effective for our purpose. In the lower left side is given Ion Ranges of 5 MeV alpha particles in watery liquid, 701. Alpha particle energy loss by ionization, 702, is plotted in upper left side showing its path through 5 microns of Lexan, 2 microns of gold and the rest is deposited in water. Considering that every 100 eV of deposited energy breaks about 5-6 water molecules we see that every angstrom in water harvest enough energy for 1 radiolysis, while a water molecule has about 3 angstroms. That allows us to calculate the radiolysis rate, and radiation absorbed dose. Alpha particles energy deposition into recoils, 703, is shown in upper right plot that shows that each ion basically recoils and dissociates a water molecule at the end of range that adds to the radiolysis due to ionization. Alpha particle energy deposition into phonons, 704, plotted in the lower right side shows that at the end of alpha particle range temperature per water molecule may be as high as 12 eV, that correspond to about 140,000 K degrees, that corresponds to a plasma state, for a volume of about 2 microns. This is so called Bragg peak, which in water behaves as a micro-cavitation. Here we estimate that about 1 million of molecular bounds are broken per each particle, and because ICF has only few percent of organic matter probability those organic matter radicals to be terminated short with water free radicals is high, driving to decrease in viscosity and probability of clogging on plastic internal surfaces of the shunt.

FIG. 8 shows some valve shutter mechanism details, where the valve wall is presented as valve's separation structure, 800, having a liquid intake cavity, 801, and a liquid exhaust cavity, 802, separated by valve's seat, 803, and valve's lid, 806, actuated by valve lid's rod, 807, that has a protective bellow cover, 808, that may be immune to cerebrospinal fluid (CSF) deposition. When the intracranial pressure (ICP) grows over acceptable limit, valve opens making liquid intake flow, 804, pass through the space between valve's lid, 806 and valve's sit, 803, and forms the liquid output flow, 805, towards the stomach, making intracranial pressure (ICP) decrease. The most sensitive parts to clogging are the valve's lid surface that is treated and turns into a radioactive surface, 809, and sit's shutting surface that becomes radioactive sit surface, 810, preventing both clogging and infections in the area, because radiation also damages bacteria and virioli, and not only organic residues in the cerebrospinal fluid (CSF).

FIG. 9 shows the radioactive layer structure for alpha emitter implantation, where material bulk, 900, is first implanted with a layer to stop diffusion in bulk, 901, than radioactive isotope, 902, is added at a lower depth, and over it towards the surface a layer to stop diffusion outside the bulk, 903, is implanted, such as to lock in place the radioactive emitter material. A chemical inert layer, 904, is deposited to further improve functionality and interaction with hydrocephalic liquid, 905, and minimize wear, corrosion or chemical interaction and organic matter deposition.

Figure also shows how radiation split molecular bound, 907, of any organic molecule, 906, where beta radiation may be passing without interaction, 908, having a large path than alpha particles, 909, in an organic matter dominated by water molecules, 910, splitting them in shorter radicals.

FIG. 10 shows a shunt with Wi-Fi communication, made of an actual shunt, to which a shunt's programing and control unit, 1000, have been added, containing a pressure measuring transducer, 1001, valve position monitoring, 1002, battery, 1003, pressure regulator shunt, 1004, communication and control micro-processor, 1005, that uses an RF signal, 1006, to with outside body equipment, that may be a dedicated device, cell-phone or computer. The shunt has its usual liquid input, 1007, in the first cavity, 1008, and from there in the valve cavity, 1009, then passing through valve seat, 1010, when valve is open through shunt's exit tube, 1011, towards stomach.

Private industry would be employed to build the many units required as accessories to form a new product addressing these most critical situations. It was conceived to keep the cost as low as possible, to be largely accessible, and make a drastic improvement in the way the most important part of the sickness cycle is treated. Being equipped with an expert program, it will make a difference, in sickness assistance, predicting the need for emergency care, in the situations when medication and reprograming is inefficient, being possible to connect in real-time with physician, and seek emergency response, or treating a disease in ambulatory conditions.

### EXAMPLES OF THE INVENTION

Thus it will be appreciated by those skilled in the art that the present invention is not restricted to the particular preferred embodiments described with reference to the drawings, and that variations may be made therein without departing from the scope of the present invention as defined in the appended claims and equivalents thereof. The present invention consists in development of a method to implant radioactive isotopes in the critical technologic surfaces of the shunt, to improve its good operation duration, and using Wi-Fi connected local embedded data acquisition (daq) system to have compressive bio-metric and medical evaluation and improve the research data base with new reliable information.

The invention may be also applied in very complex situations, allowing the users to get complex data, as for scientific purposes or to test new prototypes.

The present invention relies on the customization of the data acquisition equipment to serve the most urgent needs, fulfilling the gap between computer simulators and real life, where patient's biometric evaluation in possible simultaneously with the normal operation.

What is claimed is:

1. A method to improve the duration of good operation of hydrocephalus shunts comprising of: a. A shunt part before assembly with critical technologic surfaces accessible for ion beam;
b. An implantation of an in-depth stabilization layer to prevent alpha emitter isotope diffusion in depth (for beta emitter may be applied but it is not critical);
c. An implantation layer placed few microns from the surface in shunt's critical surfaces that allows alpha or beta radiation escape in nearby fluid stopping into it, and breaking the organic matter into smaller entities unable to deposit on the protected surfaces.
d. An implantation of an outer layer that prevents the diffusion of the radioactive isotope to the surfaces, locking it in position, and preventing it from escaping in the fluid.

2. A method to improve the duration of good operation of hydrocephalus shunts according to claim 1 that allows control of depth and radiation type and absorbed dose.

3. A method to improve the duration of good operation of hydrocephalus shunts according to claim 1, where the radioactive implanted layer is locked in depth between two layers of materials that prevents the diffusion through of the radioactive material.

4. A method to improve the duration of good operation of hydrocephalus shunts according to claim 1 where the isotope used is customized on application type of stent and disease particularities;

10. A shunt device that improves the good operation duration and allows continuous data transfer, made of:
a. An improved shunt with anti-clogging layers embedded;
b. A micro-processor embedded near shunt system comprising of:
i. Battery
ii. Micro-processor
iii. DAQ collecting information of:
1. Temperature;
2. Pressure in liquid in input,
3. Differential pressure,
4. Conductivity,
5. Other electric signals,
6. Solenoid Triggering signal and,
7. Battery voltage
iv. Wi-Fi module

11. A shunt device according to claim 10 that may use Wi-Fi to connect to an external device and exchange data.

12. A shunt device according to claim 10, where the sensitive parts are made of various materials, that are suitable for being implanted with the isotope layers and be coated with materials that are rejecting the clogging.

13. A shunt device according claim 10, where the good operation is continuously reported via a wi-fi to a local data acquisition and processing unit.

14. A shunt device according to claim 10, where anticipation for maintenance program is possible to be set based on evolution of the measured parameters.

15. A shunt device according to claim 10, where the type of radioisotope and its activity is customized for the patient.
